(19) United States
(12) Patent Application Publication
(10) Pub. No.: US 2023/0235242 A1
(43) Pub. Date: Jul. 27, 2023

(54) MICROEMULSIONS AND THEIR USES

(71) Applicant: CFLIQUIDS AB, BANDHAGEN (SE)

(72) Inventor: Per WIKLUND, Stockholm (SE)

(21) Appl. No.: 17/999,618

(22) PCT Filed: Jun. 8, 2021
(86) PCT No.: PCT/SE2021/050548

§ 371 Date: Nov. 22, 2022

(30) Foreign Application Priority Data
Jun. 9, 2020 (SE) 2050672-1

(51) Int. Cl. C10M 169/02; C10M 105/10; C10M 105/04; C10M 109/02; C10M 105/06; C10M 107/02; C10M 111/06; C10M 117/02; C09D 5/08; C09D 5/00; C09D 5/02; C09D 15/00; C09D 191/00; C04B 41/45; C04B 41/47; C04B 41/00; D21H 19/12; D21H 21/16; C14C 9/02; C10L 1/32; B01F 23/41; B27K 3/00; B27K 3/50; C23F 11/10; B27K 3/48

(52) U.S. Cl. CPC C10M 169/02; C10M 105/10; C10M 105/04; C10M 109/02; C10M 105/06; C10M 107/02; C10M 111/06; C10M 117/02; C09D 5/08; C09D 5/008; C09D 5/022; C09D 15/00; C09D 191/00; C04B 41/4539; C04B 41/472; C04B 41/009; D21H 19/12; D21H 21/16; C14C 9/02; C10L 1/328; B01F 23/4105; B01F 23/4143; B01F 23/4145; B27K 3/005; B27K 3/50; C23F 11/10; B27K 3/48; C10M 2201/02; C10M 2203/022; C10M 2203/065; C10M 2205/0206; C10M 2207/0215; C10M 2207/0225; C10M 2207/1265; C10M 2207/1296; C10M 2207/401; C10M 2205/20; C10N 2050/013

(57) ABSTRACT

The invention relates to a microemulsion comprising water in an amount of 1-30 w %; sodium or potassium oleate, Na/K salts of tall oil fatty acid, and/or Na/K salts of C16-C18 saturated or unsaturated fatty acids in an amount of 10-40 w %; oleic acid, tall oil fatty acid, or C16-C18 saturated or unsaturated fatty acids in an amount of 2-40 w %; ethanol in an amount of 0-40 w %; glycerol in an amount of 5-40 w %; and liquid hydrocarbon(s) in an amount of 5-40 w %, up to a maximum or total of components parts of 100 w %. Moreover, methods of manufacture and uses of the microemulsion are disclosed.

### FIELD OF INVENTION

The invention relates to microemulsions, and in particular to non-water-soluble microemulsions.

### BACKGROUND

Oils are efficient lubricants to reduce friction between moving parts in many mechanical devices, but oils are by nature liquids that must be contained to stay put. In contrast lubricant greases are viscoelastic materials that do not flow away unless high pressure is applied. Hence lubricant greases can be used in e.g. bearings, where they can be contained efficiently with gaskets. Lubricant greases, and high viscosity oils, are also commonly applied to moving mechanical parts that cannot be enclosed, such as driving chains and belts, wire winches, cranes, braking calipers, leaf springs, chain saw chains etc. In such applications it is inevitable that lubricant is emitted to the environment, and that the lubricant gets into contact with water or moisture. For these reasons it is preferable if the lubricant is not environmentally harmful and is not broken down or washed away by water.

Lubricant greases consist mainly of oil with a minor content of a thickener. The thickener can be fine powders, yielding greases that are suspensions. Most commonly however, oil is thickened by emulsification with metal soaps of fatty acids. The most common fatty acids used are stearic acid and 12-hydroxy-stearic acid, usually saponified with lithium or calcium as counterions. Lithium has of late found another use in electric batteries, and competition for the commodity has increased. The oils used in lubricant greases are usually mineral oils of considerable viscosity, but it is also possible to use vegetable oils at the price of poor oxidation stability and poor low temperature properties.

Microemulsions are seemingly homogenous mixtures of liquids not normally miscible with each other due to their vast difference in polarity. In the presence of surfactants such liquids can in certain cases, and in certain proportions, spontaneously form visibly clear emulsions, in particular in presence of so-called co-surfactants which are usually medium sized alcohols. Like macroemulsions, which are opaque due to larger micelle size, microemulsions can be of oil-in-water or water-in-oil types. In addition, microemulsions can appear in a third discontinuous type.

Descriptions of emulsions and microemulsions for use as lubricants are abundant in the patent literature, in particular in the field of metal working fluids where the general aim is to incorporate as much water as possible in the fluid (for the purpose of cooling) yet maintain the lubricant effect.

In general, the described types of emulsions consist of mineral oils, often of high viscosity, water (often more than 25 w %), and emulsifier(s). The content of emulsifier(s) is usually kept as low as possible as the cost for many efficient synthetic emulsifiers is relatively high. Hence, there is a need for new microemulsions reducing the need for costly emulsifiers, and reducing the environmental impact through the use of oils or hydrocarbons of non-fossil origin.

### Short Description of the Invention

The invention relates to a microemulsion comprising: water in an amount of 1-30 w %; sodium or potassium oleate, Na/K salts of tall oil fatty acid, and/or Na/K salts of C16-C18 saturated or unsaturated fatty acids in an amount of 10-40 w %; oleic acid, tall oil fatty acid, or C16-C18 saturated or unsaturated fatty acids in an amount of 2-40 w %; ethanol in an amount of 0-40 w %; glycerol in an amount of 5-40 w %; and liquid hydrocarbon(s) in an amount of 5-40 w %, up to a maximum or total of components parts of 100 w %.

The microemulsion is prepared using a method comprising the following consecutive steps: (a) mixing of NaOH or KOH with water, glycerol, and ethanol to obtain a solution; (b) mixing oleic acid, tall oil fatty acid, and/or C16-C18 saturated or unsaturated fatty acid(s) into the solution to obtain a microemulsion; (c) mixing of the microemulsion with liquid hydrocarbon(s) to form a second microemulsion; (d) optionally addition of water or ethanol; and (e) optionally full or partial evaporation of the ethanol, whereby steps (d) and (e) may be performed in reversed order.

The microemulsion finds its use as e.g. a lubricant, metal working fluid, water proofing agent for porous materials, and in in pharmaceutical preparations.

The microemulsions according to the invention comprise low viscosity hydrocarbon fluids (biobased or of fossil origin), high amounts of natural emulsifiers, low amounts of water, and glycerol. The innovative microemulsions as described herein possess a unique specific property in that they form a non-soluble material when contacted with excess water. That is to say, they cannot be referred to as soluble oils which is a common term for oil preparations forming emulsions when mixed with water.

The microemulsions disclosed herein exist as microemulsions at ambient temperature, unless another temperature is specifically disclosed. In certain embodiments, microemulsions exist as microemulsions only at a temperature exceeding 60° C., e.g. in a temperature interval from 60 to 90° C. Microemulsions comprising palmitic and/or stearic acid are examples of the latter kind of microemulsions.

### DETAILED DESCRIPTION OF THE INVENTION

It has unexpectedly been found that certain homogenous clear mixtures of water, water soluble alcohols, sodium oleate, oleic acid and glycerol, form homogenous mixtures when blended with non-polar liquids such as hydrocarbon solvents and oils.

Furthermore, the amount of such non-polar liquids that can be added to the original mixture, still yielding a clear homogenous mixture, can be increased by the addition of more oleic acid. These homogenous mixtures are microemulsions, i.e. thermodynamically stable emulsions that spontaneously form without the need for high shear mixing (the process of homogenizing otherwise immiscible liquid phases) associated with the formation of ordinary opaque (macro) emulsions.

Depending on the composition and order of addition of the liquid components, these microemulsions behave differently when diluted with more water. In certain cases, were the amount of water present when the microemulsion is formed is small, dilution with water yields a fluffy or crystalline-like material which does not dissolve in water. In other cases, in particular when the amount of water present during the formation of the microemulsion is higher, dilution with water yields soapy emulsions easily washed away with water.

It was observed that when a solution of oleic acid (54 g) and hexadecane (33 g) was added by slight stirring to a mixture of aqueous sodium hydroxide (18 g 33% NaOH), glycerol (69 g) and ethanol (60 g 95%), an opaque mixture was formed. Upon addition of a portion of oleic acid (5 g) the mixture became a clear microemulsion. Only 42 g of oleic acid was needed to neutralize the sodium hydroxide, meaning that about 17 g of oleic acid, which is oil soluble, resides in the mixture. Another addition of hexadecane (10 g) again made the mixture opaque, and a further addition of oleic acid (5 g) again produced a microemulsion. This scheme of gradual addition of the two components could be continued with more hexadecane (up to at least another 35 g) and more oleic acid (up to at least 10 g) to maintain the microemulsion state of the system. When a portion of the final microemulsion (2.5 g) was poured on water (100 g), a fluffy white crystalline-like material was formed which had the consistency of a soft lubricant grease. When smeared on fingers this material was not possible to wash away with water without the use of a detergent.

To further analyze the behavior of this system, five different experimental mixtures were prepared, see Table 1.

TABLE 1

| Case | 1 | 2 | 3 | 4 | 5 |
|---|---|---|---|---|---|
| NaOH (g) | 6 | | 6 | 6 | 6 |
| Water (g) | 12 | 12 | 12 | 12 | 60 |
| Glycerol (g) | 70 | 70 | 130 | | 70 |
| Ethanol (g) | 60 | 60 | | 130 | 60 |
| Oleic acid (g) | 70 | 70 | 70 | 70 | 70 |
| Description of | Homogenous | Opaque | Thick | Homogenous | Homogenous |
| mixture | clear liquid | separating | slightly | clear liquid | clear liquid |
| | | mixture | opaque | | |
| | | | mixture | | |

In Case 1, a solution of sodium hydroxide, a small quantity of water (deionized), glycerol and ethanol (95%), formed a homogenous clear microemulsion upon light stirring wherein the remaining oleic acid not neutralized by sodium hydroxide (to form sodium oleate) constitutes the oil phase. Whereas sodium oleate has a limited solubility in water, oleic acid is practically insoluble in water. And although the protonated carboxy group of oleic acid is in itself polar, it is not polar enough for (non-ionized) oleic acid to behave as a surfactant. Hence, as shown in Case 2, in the absence of the strong base sodium hydroxide, no emulsion was formed.

If the portion of ethanol is fully replaced by glycerol, as in Case 3, the resulting mixture appears homogenous, albeit not fully clear. If glycerol is fully replaced by ethanol, as in Case 4, the mixture forms a clear microemulsion. It was also found that a microemulsion was formed when more water was used to dissolve the sodium hydroxide, as shown in Case 5.

The microemulsions of Cases 1, 3, 4 and 5 all behave similar to washing soap when mixed with water, i.e. they lather and can easily be rinsed away with water.

In Case 1 it was possible to add a portion of hexadecane (70 g) to form a new second generation clear stable microemulsion. Hexadecane (70 g) could also be added in Case 3, but the resulting emulsion was only formed after vigorous shaking and was not entirely clear. Upon storage for several weeks this very viscous emulsion started to separate, leading to the conclusion that it was not a true microemulsion (which would be stable). In Case 5, a portion of up to 25 g of hexadecane could be added leading to formation of a second generation microemulsion.

Hence the presence of glycerol is necessary for the second generation microemulsions to form. The presence of ethanol is not necessary for formation of second generation emulsions in general (after addition of hexadecane) but appears to be necessary for the formation of a (stable and visibly clear) microemulsion and also contributes to ease of blending of the components as its presence markedly reduces viscosity.

The ability of oleic acid/sodium oleate in water or glycerol, to form emulsions with hexadecane, is known [1], but apparently only macroemulsions were studied, since no mention of microemulsions was made and high energy mixing by sonication was used to prepare the white emulsions. The authors did not report any use of ethanol or other cosolvents, nor the presence of both glycerol and water simultaneously.

Microemulsions consisting of sodium oleate/oleic acid, water and isopropanol have also been reported [2], but further addition of hydrocarbon solvents or oils to the mixtures has not been disclosed.

It was found that two of these three second generation microemulsions (stemming from Cases 1 and 3) gave the grease-like material when poured on water (cold or hot). The sticky material could not be washed away or dissolved in much larger quantities of water.

This behavior is surprising for several reasons. Sodium soaps of fatty acids are generally not useful as thickeners in lubricating greases because they are water soluble (compare ordinary washing soap) and give greases that are easily dissolved and washed away by water [3]. Furthermore, production of water-resistant lubricating greases by conventional methods requires oils of much higher viscosity and hence molecular size than the C16-structure of hexadecane. Lastly, oleic acid, although extremely abundant in natural fats and oils, is not normally thought of as a basic component of lubricating greases.

A microemulsions displaying the behavior of formation of grease-like floccular material when poured on water will in the following be referred to as a Flocculation Forming Microemulsion (FFMe).

When the emulsion stemming from Case 5 was poured on water it instead gave a soapy material easily washed away with water. This microemulsion, initially containing much more water, is likely an oil-in-water type microemulsion, whereas the FFMe stemming from Case 1 is likely to be a water-in-oil microemulsion. A further observation was that the latter FFMe could be diluted with at least 60 g of (deionized) water and still retain the FFMe characteristics.

The invention shall now be described with reference to the below examples, which shall not be seen as any limitation on the claimed scope whatsoever. The person skilled in the art realizes that exchange of component parts and change of constitution may be made, without departing from the inventive concept.

### Delineating Characteristics

To further delineate important characteristics of the above described microemulsions leading to the flocculation forming properties, a series of experiments were performed. In the following the definition of an FFMe is that when 3 ml of the microemulsion is poured on 100 ml water, slight swirling of the vessel containing the mixture generates a floating (white) floccular material within 2 hours. In cases of strong FFMe-effect the material formed is granular or clearly lumpy.

TABLE 2

| Case# | 6 | 7 | 8 | 9 | 10 | 11 | 12 | 13 | 14 |
|---|---|---|---|---|---|---|---|---|---|
| NaOH (g) | 6 | 6 | 6 | 6 | 6 | 6 | 6 | 6 | 6 |
| Water (g) | 12 | 12 | 12 | 12 | 12 | 12 | 12 | 12 | 12 |
| Glycerol (g) | 20 | 20 | 20 | 40 | 60 | 60 | 20 | 20 | 60 |
| EtOH (g) | 20 | 20 | 20 | 20 | 60 | 60 | 60 | 30 | 60 |
| Oleic Acid (g) | 50 | 60 | 60 | 60 | 60 | 70 | 70 | 70 | 70 |
| Hexadecane (g) | 10 | 10 | 30 | 30 | 30 | 30 | 30 | 30 | 10 |
| Clear | No | Almost | Yes | Yes | Yes | Yes | Yes | No | Yes |
| FFMe-effect | No | No | No | Slight | No | Strong | Moderate | No | No |

In all cases above (6-14) the initial mixing of NaOH, water, glycerol and ethanol gives a clear solution. When exchanging ethanol for isopropanol this was not found to be the case even if the final microemulsions after addition of hexadecane did form. However, the isopropanol containing microemulsions did not show FFMe-behavior in any case.

Microemulsions could also be prepared with Rape seed methyl ester (RME/FAME) and vegetable oils, but these did not show the FFMe-effect.

### EXAMPLES

Using the methodology described above, a series of FFMe:s were prepared. Table 3 shows examples of FFMe:s based on hexadecane. The viscosity of the fluid in Example 1 was found to be 30 cp at 20° C., and the viscosity of the fluid in Example 2 was found to be 65 cp at 20° C.

TABLE 3
Hexadecane-based FFMe:s

| Example | 1 | 2 | 3 | 4 | 5 | 6 | 7 |
|---|---|---|---|---|---|---|---|
| Water w % | 5 | 5 | 9 | 6 | 3 | 8 | 7 |
| Ethanol w % | 21 | 19 | 12 | 25 | 26 | 25 | 30 |
| Glycerol w % | 24 | 31 | 24 | 25 | 26 | 25 | 10 |
| Sodium Oleate w % | 16 | 14 | 27 | 19 | 20 | 19 | 23 |
| Oleic Acid w % | 10 | 10 | 10 | 12 | 12 | 11 | 14 |
| Hexadecane w % | 24 | 22 | 18 | 13 | 13 | 12 | 15 |

Table 4 shows five examples where HVO (hydrotreated vegetable oil) is used as a component of FFMe:s. HVO is most commonly used as a high quality renewable diesel, but can also be used as a chemical component of various functional fluids. Because of its chemical origin in fatty acids, HVO is dominated by C16-C18 hydrocarbons, but unlike hexadecane it is highly isomerized. Example 10 shows that potassium oleate works as well as sodium oleate.

As stated above rape seed methyl ester (RME/FAME) and vegetable oils can give stable microemulsions, but these did not show the FFMe-effect. However, an FFMe containing a combination of HVO and rapeseed oil could be formulated (Example 11), and similarly a formulation with linseed oil was made to which a portion of wood tar could be added with retained FFMe-behavior (Example 12). The latter example shows that the FFMe-formulations can dissolve other components and incorporate them into the microemulsion.

The microemulsion of Example 6 could be further diluted with up to 35 w % water or 30 w % ethanol without compromising the microemulsion state of the preparation. At higher dilution, translucency ceased and eventually the mixture formed several liquid phases.

TABLE 4
HVO-based FFMe:s. UFA is short for C16-C18 unsaturated fatty acids (CAS# 67701-
08-8). HVO is short for hydrotreated vegetable oil (diesel type fraction), REACH-registration
number 01-2119450077-42-0000.

| Example 8 | Example 9 | Example 10 | Example 11 | Example 12 |
|---|---|---|---|---|
| 6 w % water | 6 w % water | 6 w % water | 7 w % water | 7 w % water |
| 23 w % ethanol | 23 w % ethanol | 23 w % ethanol | 20 w % ethanol | 18 w % ethanol |
| 26 w % glycerol | 26 w % glycerol | 26 w % glycerol | 19 w % glycerol | 18 w % glycerol |
| 20 w % Sodium | 20 w % Sodium | 20 w % Potassium | 22 w % Sodium | 21 w % Sodium |
| Oleate | UFA salt | Oleate | UFA salt | UFA salt |
| 12 w % oleic acid | 12 w % UFA | 12 w % oleic acid | 13 w % UFA | 13 w % UFA |
| 13 w % HVO | 13 w % HVO | 13 w % HVO | 12 w % HVO | 11 w % HVO |
| | | | 7 w % rapeseed oil | 7 w % linseed oil |
| | | | | 5 w % wood tar |

Further examples of FFMe:s based on other hydrocarbon liquids are found in Table 5.

Noteworthy is that microemulsification of the PAO-based engine oil 5W-30 requires more free oleic acid. This is a parallel to the initial observation that higher amounts of hexadecane could be microemulsified with increased addition of oleic acid.

TABLE 5
Further FFMe:s. UFA is short for C16-C18 unsaturated fatty acids (CAS# 67701-08-8).
ISA is short for C11-C15 isoalkanes (CAS# 90622-58-5). T9 is a napthenic base oil of viscosity
9 mm²/s at 40° C. (Nynas AB). 5W-30 is a formulated PAO-based engine oil.

| Example 13 | Example 14 | Example 15 | Example 16 | Example 17 |
|---|---|---|---|---|
| 6 w % water | 7 w % water | 5 w % water | 7 w % water | 5 w % water |
| 23 w % ethanol | 21 w % ethanol | 22 w % ethanol | 10 w % ethanol | 7 w % ethanol |
| 26 w % glycerol | 20 w % glycerol | 22 w % glycerol | 28 w % glycerol | 31 w % glycerol |
| 20 w % Sodium | 23 w % Sodium | 16 w % Sodium | 23 w % Sodium UFA | 16 w % Sodium |
| UFA salt | UFA salt | Oleate | salt | UFA salt |
| 12 w % UFA | 14 w % UFA | 24 w % oleic acid | 14 w % UFA | 16 w % UFA |
| 13 w % ISA | 15 w % T9 | 11 w % 5W30 | 18 w % HVO | 25 w % HVO |

### Use of Additional Fatty Acids

In a variant of the preparation of Example 16, 18 g of aqueous sodium hydroxide (33%) was diluted with 50 g of ethanol and 55 g of glycerol, after which 70 g of Tall Oil Fatty Acids (TOFA) was mixed in, followed by addition of 35 g of HVO. This procedure gave a dark colored microemulsion. The contents of TOFA are dominated by C18 unsaturated fatty acids.

In yet another variant of Example 16, 18 g of aqueous sodium hydroxide (33%) was diluted with 30 g of ethanol and 60 g of glycerol after which a solution of 10 g of palmitic acid in 60 g of oleic acid was mixed in together with 35 g of hexadecane to form a microemulsion.

In yet another variant of Example 16, 18 g of aqueous sodium hydroxide (33%) was diluted with 30 g of ethanol and 60 g of glycerol, after which 70 g molten palmitic or stearic acid mixed with 35 g of hexadecane was added under vigorous stirring. While the resulting preparation was warm, it showed the same microemulsion characteristics as all of the above examples, but when cooled to room temperature the preparation solidified into a uniform wax like material with a melting point of about 60° C.

### Behavior of the Flocculation Forming Microemulsions (FFMEs)

The floccular/granular grease-like material formed when the microemulsions were poured on proportionally high amounts of water was possible to capture in a sieve of mesh size 1 mm at room temperature upon which the sieve was clogged and no further flow through it occurred.

When the microemulsions were mixed with about equal volumes of water, thick creamy emulsions formed. These emulsions were found to be stable over a period of several days, but as water (and ethanol) evaporated the material gradually changed into a clear oil-like substance of high viscosity.

When the ethanol in microemulsion of Example 8 was allowed to fully evaporate, what resulted was a very viscous oil-like substance with a density of 0.97 g/ml at 20° C., and a dynamic viscosity at 40° C. of around 220 cp, indicating a kinematic viscosity of about 227 mm²/s at 40° C. The substance was immiscible with water, but gradually formed a similar grease-like material as in the cases when an FFMe was poured on water (see above), but of a lumpier texture. Furthermore, when the microemulsion of Example 16 was heated to 80° C. until bubbling had subsided and the ethanol had been evaporated, a microemulsion of very high viscosity remained (987 cp at 20° C.). The high viscosity microemulsion retained its stability over a period of 3 months. In a subsequent step, the new microemulsion was heated to 100° C. until bubbling had subsided and the water had been evaporated, whereby a microemulsion of viscosity 565 cp at 20° C. remained.

The microemulsion of Example 16 may be used as a thickener of HVO. When 50 w % of the said microemulsion was shaken with HVO, a gel-like substance was produced with a viscosity of 52 cp at 20° C. Similarly, 35% w % of the microemulsion yielded a gel-like substance of viscosity 22 cp at 20° C. The gelated HVO may be used as a fuel or as a lubricant.

### Applications of the Microemulsions

Because of the grease-like behavior of these microemulsions after contact with water, they can be used for lubrication of open mechanical systems exposed to the weather such as the sliding parts of hydraulic booms and cranes, wires and winches, driving belts and chains, chain saw chains, sliding tables or ramps etc. This type of equipment is very common on ships' decks. In the wet the material would act similarly to common lubricant grease, and when allowed to dry out the material instead resembles a high viscosity oil.

Another application for the microemulsions are as metal working fluids. In all these lubricant cases it is suitable to additivate the liquid to preserve the function over time with phenolic (such as BHT) and/or aromatic amine antioxidants, and to improve lubricity with antiwear additives (such as zink dithiophosphates and glycerol monooleate) and extreme pressure additives (such as dithiocarbamates).

The microemulsions can be used to protect metal surfaces from corrosion. A plate mild steel was dipped into the microemulsion of Example 8, after which the plate was placed in a beaker containing an aqueous solution of sodium chloride (4%). As comparison a plate from the same sheet of steel was also placed in the same solution. After 48 hours the plates were lifted from the brine solution. The treated plate was still covered by the grease-like layer. The plates were washed with detergent and clean water. Upon inspection after the cleaning the untreated plate was found to have very clear corrosion scars whereas the treated plate was in pristine condition. Easily removable layered corrosion protection is often referred to as temporary corrosion protection and is useful e.g. during storage (hours to months) of partially processed metal parts to be ready for the next stage of production.

It was found that when emulsions of the invention are painted onto raw concrete surfaces, such as e.g. concrete slabs, what results is a persistent water repellant surface. If a drop of water is applied to a raw concrete surface it is normally instantly absorbed into the material. In contrast, a drop of water applied to a raw concrete surface treated with a microemulsion of the invention stays intact and rolls off upon tilting. Larger amount of water sprayed onto the treated concrete surface triggers the formation of the grease-like material making the surface sticky. As the experiment with steel plates shows, the sticky grease-like layer is impenetrable to water. In combination with the observation that the floccular material formed when the FFMe:s are poured on water, it is envisioned that they can function as water proofing agent on porous materials like rock or concrete.

It was also found that when paper, paperboard or cardboard was coated with a thin layer, e.g. 1-100 μm, of the innovative microemulsion, the coating provided a measure of waterproofing. Hence, the microemulsions are useful e.g. as barrier for packaging materials.

In a similar fashion the FFMe:s can be used for impregnation of wood, both to make it water-repellant and more resistant to rot. An FFMe with a dissolved drying (or non-drying) vegetable oil and wood tar, possibly in combination with conventional wood preservatives including sulfur, silicates, copper salts/complexes or (suspended) metallic copper particles and methylisothiazolinones may be produced. In an exemplary embodiment, two dimensionally identical pieces (75×17×195 mm) of Nordic spruce was cut from the same plank at the same time, ensuring equal moisture content. One of the pieces was put horizontally with the largest area down in a container with a layer of the microemulsion of Example 12 reaching about 5 mm up the side of the wooden piece. After 24 h the piece was turned to soak the other side. After another 24 h the piece was lifted from the bath and allowed to dry. The two pieces of wood were stored indoors for one week after which weighing showed an increase in weight for the treated piece by 4% compared to the untreated piece. When sawn apart, the cross-section of the treated piece showed the liquid had penetrated about 2-5 mm into the wood, and the surface of the wood had fatty feel to it even after rinsing with water and letting dry. In a study from 2006 [4] it was concluded that (macro) emulsions of tall oils can be used efficiently to impregnate wood. The limiting factor was found to be that the relatively large micelles of the emulsions was filtered by the cellulose of the wood, and hence deep penetration was difficult. A microemulsion of the invention would suffer much less from this problem, in particular if the viscosity is kept low with the content of ethanol.

It was found that the microemulsions may be used as an alternative to silicone-based products to rejuvenate and add shine to rubber surfaces such as car tires. Similarly, the microemulsions find use as a leather shine and impregnation fluid.

### LITERATURE REFERENCES

[1] “Self-assembly and emulsions of oleic acid-oleate mixtures in glycerol”, Delampe, Jerome, Barrault, Douliez; Green Chemistry, 2011 (13), 64-68
[2] “Conversion of a surfactant-based microemulsion to a surfactant-free microemulsion”; Liu, Lu, Zhang, Zhu, Huang; Soft Matter, 2019 (15), 462-469
[3] Chemistry and Technology of Lubricants, 3:rd edition: ed. Mortier, Fox, Orszulik: Springer, Dordrecht Heidelberg, London, New York: 2010: p. 420
[4] “Tall oil/water—emulsions as water repellents for Scots pine sapwood” Hyvönen, Piltonen, Niinimäki, Holz als Roh- and Werkstoff, 2006 (64), 68-73

### The Invention in Bullet Points

1. Microemulsion comprising: water in an amount of 1-30 w %; sodium or potassium oleate, Na/K salts of tall oil fatty acid, and/or Na/K salts of C16-C18 saturated or unsaturated fatty acids in an amount of 10-40 w %; oleic acid, tall oil fatty acid, or C16-C18 saturated or unsaturated fatty acids in an amount of 2-40 w %; ethanol in an amount of 0-40 w %; glycerol in an amount of 5-40 w %; and liquid hydrocarbon(s) in an amount of 5-40 w %, up to a maximum or total of components parts of 100 w %.

2. Microemulsion according to claim 1, comprising: water in an amount of 2-10 w %; sodium or potassium oleate, Na/K salts of tall oil fatty acid, and/or Na/K salts of C16-C18 saturated or unsaturated fatty acids in an amount of 14-27 w %; oleic acid, tall oil fatty acid, or C16-C18 saturated or unsaturated fatty acids in an amount of 10-24 w %; ethanol in an amount of 0-40 w %, e.g. 0-26 w %; glycerol in an amount of 10-31 w %; and liquid hydrocarbons in an amount of 11-24 w %, up to a maximum or total of components parts of 100 w %.

3. Microemulsion according to any of the preceding claims, wherein the liquid hydrocarbon is C11-C20 alkanes and/or isoalkanes.

4. Microemulsion according to any of the preceding claims, wherein the liquid hydrocarbon is hexadecane, diesel, HVO, PAO, a naphthenic or paraffinic base oil or a combination thereof.

5. Microemulsion according to any of the preceding claims, having a viscosity in the interval of from 20 to 400 cp at 20° C., e.g. 50 to 200 cp at 20° C.

6. Microemulsion according to any of the preceding claims, having a storage stability of more than 30 days at 20° C.

7. Microemulsion according to any of the preceding claims, to which wood tar has been added in an amount of up to 15 w %.

8. Microemulsion according to any of the preceding claims, to which an antioxidant, antiwear agent, and/or extreme-pressure additive has been added in an amount of 0.1 to 5 w %.

9. Microemulsion according to any of the preceding claims, to which wood preservatives such as antifungal substance(s) have been added in an amount of 0.1 to 5 w %.

10. Microemulsion according to any of claims 1-11, comprising 5-40 w % ethanol and existing in the form of a microemulsion.

11. Microemulsion according to claim 10, comprising 12-26 w % ethanol.

12. Gel, comprising 35-50 w %, e.g. 40 or 45 w %, or any interval thereinbetween, of the microemulsion according to any of claims 1-11, and 50-65 w %, e.g. 55 or 60 w %, or any interval thereinbetween, of a hydrotreated vegetable oil, up to a maximum or total of components parts of 100 w %.

13. Method of preparing a microemulsion according to any of claims 1-11, comprising the following consecutive steps: (a) mixing of NaOH or KOH with water, glycerol, and ethanol to obtain a solution; (b) mixing oleic acid, tall oil fatty acid, and/or C16-C18 saturated or unsaturated fatty acid(s) into the solution to obtain a microemulsion; (c) mixing of the microemulsion with liquid hydrocarbon(s) to form a second microemulsion; (d) optionally addition of water or ethanol; and (e) optionally full or partial evaporation of the ethanol, whereby steps (d) and (e) may be performed in reversed order.

14. Method according to claim 13, wherein in step (d) water or ethanol is added.

15. Method according to claim 13, wherein in step (e) ethanol is fully or partially evaporated.

16. Method according to any of claims 13-15, wherein all carbon compounds are biogenic.

17. Use of a microemulsion according to any of claims 1-11 for temporary corrosion protection on metal surfaces, especially steel.

18. Use of a microemulsion according to any of claims 1-11 as a lubricant.

19. Use according to claim 18, wherein driving chains, chain saw chains, belts, wire winches, cranes, brake calipers, chain saw chains, or leaf springs are lubricated.

20. Use of a microemulsion according to claims 1-11 as metal working fluid.

21. Use of a microemulsion according to any of claims 1-11 as a water proofing agent for porous materials.

22. Use according to claim 21, wherein the porous materials constitute rock cavities and/or concrete constructions.

23. Use of a microemulsion according to any of claims 1-11 for coating of paper, paperboard, cardboard, or rubber.

24. Use of a microemulsion according to any of claims 1-11 as a leather or rubber shine liquid, or as a leather impregnation liquid.

25. Use of a microemulsion according to any of claims 1-11, as a thickening agent for hydrotreated vegetable oil.

26. Use of a gel according to claim 12, as a fuel or a lubricant.

27. Package or container comprising a microemulsion according to any of claims 1-11, or a gel according to claim 12, wherein the microemulsion or gel is fully diluted or intended to be diluted with up to 35 w % water.

28. Kit of parts, comprising: (a) Package or container comprising a microemulsion or gel according to claim 27; and (b) Instructions for use.

29. Wood block, characterized in that it has been impregnated with a microemulsion according to any of claims 1-11.

1. A microemulsion comprising:
water in an amount of 1-30 w %;
sodium or potassium oleate, Na/K salts of tall oil fatty acid, and/or Na/K salts of C16-C18 saturated or unsaturated fatty acids in an amount of 10-40 w %;
oleic acid, tall oil fatty acid, or C16-C18 saturated or unsaturated fatty acids in an amount of 2-40 w %;
ethanol in an amount of 5-40 w %;
glycerol in an amount of 5-40 w %; and
liquid hydrocarbon(s) in an amount of 5-40 w %, up to a total of components parts of 100 w %.

2. The microemulsion according to claim 1, comprising:
water in an amount of 2-10 w %;
sodium or potassium oleate, Na/K salts of tall oil fatty acid, and/or Na/K salts of C16-C18 saturated or unsaturated fatty acids in an amount of 14-27 w %;
oleic acid, tall oil fatty acid, or C16-C18 saturated or unsaturated fatty acids in an amount of 10-24 w %;
ethanol in an amount of 5-40 w %;
glycerol in an amount of 10-31 w %; and
liquid hydrocarbons in an amount of 11-24 w %, up to a total of components parts of 100 w %.

3. The microemulsion according to claim 1, wherein the liquid hydrocarbon is C11-C20 alkanes and/or isoalkanes.

4. The microemulsion according to claim 1, wherein the liquid hydrocarbon is hexadecane, diesel, HVO, PAO, a naphthenic or paraffinic base oil or a combination thereof.

5. The microemulsion according to claim 1, having a viscosity in the interval of from 20 to 400 cp at 20° C.

6. The microemulsion according to claim 1, having a storage stability of more than 30 days at 20° C.

7. The microemulsion according to claim 1, to which wood tar has been added in an amount of up to 15 w %.

8. The microemulsion according to claim 1, to which an antioxidant, antiwear agent, and/or extreme-pressure additive has been added in an amount of 0.1 to 5 w %.

9. The microemulsion according to claim 1, to which wood preservative(s) have been added in an amount of 0.1 to 5 w %.

10. The microemulsion according to claim 1, comprising 5-40 w % ethanol and existing in the form of a microemulsion.

11. The microemulsion according to claim 10, comprising 12-26 w % ethanol.

12. A gel comprising 35-50 w % of the microemulsion according to claim 1, and 50-65 w % of a hydrotreated vegetable oil, up to a total of components parts of 100 w %.

13. A method of preparing the microemulsion according to claim 1, comprising the following consecutive steps:
(a) mixing of NaOH or KOH with water, glycerol, and ethanol to obtain a solution;
(b) mixing oleic acid, tall oil fatty acid, and/or C16-C18 saturated or unsaturated fatty acid(s) into the solution to obtain a first microemulsion;
(c) mixing of the first microemulsion with liquid hydrocarbon(s) to form a second microemulsion;
(d) optionally adding water or ethanol; and
(e) optionally partially evaporating the ethanol,
whereby steps (d) and (e) may be performed in reversed order.

14. The method according to claim 13, wherein in step (d) water or ethanol is added.

15. The method according to claim 13, wherein in step (e) the ethanol is partially evaporated.

16. The method according to claim 13, wherein all carbon compounds added to prepare the microemulsion are biogenic.

17. A method of use of the microemulsion according to claim 1, comprising using the microemulsion for temporary corrosion protection on metal surfaces.

18. A method of use of the microemulsion according to claim 1, comprising using the microemulsion as a lubricant.

19. The method of claim 18, wherein driving chains, chain saw chains, belts, wire winches, cranes, brake calipers, chain saw chains, or leaf springs are lubricated.

20. A method of use of the microemulsion according to claim 1, comprising using the microemulsion as metal working fluid.

21. A method of use of the microemulsion according to claim 1, comprising using the microemulsion as a water proofing agent for porous materials.

22. The method of claim 21, wherein the porous materials constitute rock cavities and/or concrete constructions.

23. A method of use of the microemulsion according to claim 1, comprising using the microemulsion for coating of paper, paperboard, cardboard, or rubber.

24. A method of use of the microemulsion according to claim 1, comprising using the microemulsion as a leather or rubber shine liquid, or as a leather impregnation liquid.

25. A method of use of the microemulsion according to claim 1, comprising using the microemulsion as a thickening agent for hydrotreated vegetable oil.

26. A method of use of the microemulsion according to claim 1, comprising using the microemulsion as a fuel or a lubricant

27. A package or container comprising the microemulsion according to claim 1, wherein the microemulsion is fully diluted or intended to be diluted with up to 35 w % water.

28. A kit of parts, comprising
(a) the package or container containing a microemulsion according to claim 27; and
(b) instructions for use.

29. A wood block, characterized in that it has been impregnated with the microemulsion according to claim 1.
